(19) United States
(12) Patent Application Publication
(10) Pub. No.: US 2006/0089702 A1
(43) Pub. Date: Apr. 27, 2006

(54) Sleeve to protect ratcheting stent from interference with guide catheter

(75) Inventor: Marvin J. Cervantes, Edmonton (CA)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/974,472

(22) Filed: Oct. 27, 2004

(51) Int. Cl. A61F 2/06

(52) U.S. Cl. 623/1.11

(57) ABSTRACT

A system for delivering a stent having a ratchet mechanism includes a delivery catheter having a movable elastic sleeve. The elastic sleeve covers the ratchet mechanism of the stent and prevents the ratchet mechanism from contacting a guide catheter or the vascular wall, and facilitates retraction of the delivery catheter into a guide catheter. The sleeve is retracted in order to deploy the stent. One embodiment of the invention includes a guide catheter having a curved, flexible distal tip to facilitate delivery of the stent. Another embodiment of the invention includes a method of repositioning and deploying a stent having a ratchet mechanism within a blood vessel.

### FIELD OF THE INVENTION

This invention relates generally to catheter deployment of stents. More specifically, the invention relates to a system and method for deploying stents having a ratchet mechanism, while preventing interference between the stent and a guide catheter or the vessel wall.

### BACKGROUND OF THE INVENTION

Balloon catheters are used in a variety of medical therapeutic applications including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. After inflation, the pressurized balloon exerts a compressive force on the lesion thereby increasing the inner diameter of the affected vessel and improving blood flow. Soon after the procedure, however, a significant proportion of treated vessels re-narrow due to a process called restenosis.

To prevent restenosis, short flexible mesh cylinders known as stents, constructed of metal or various polymers, are implanted within the vessel to maintain lumen size. Balloon-expandable stents are mounted on the periphery of the collapsed balloon portion of a balloon catheter at a diameter smaller than when deployed. During angioplasty, the balloon catheter carrying the stent is advanced through a network of tortuous blood vessels to the desired site. The balloon is inflated and expands the stent to a final diameter. After deployment, the stent remains in the vessel, the balloon is deflated, and the catheter is removed.

Although widely used, balloon catheters have significant limitations as stent delivery devices. The stent must be firmly attached to the exterior of the balloon, so that it does not become dislodged as the catheter passes through the vascular system to the target site. For this purpose, the stent is crimped to a sufficiently small diameter so that it grips the balloon. The shape of the balloon may be used to help secure the stent. Some catheter designs include sleeves that cover the ends of the stent, and stabilize it during passage through the vascular system.

Stents have been disclosed that are formed by rolling a flat sheet of material into a cylindrical form. When tightly rolled, the stent thus formed has a sufficiently small diameter so that it can be mounted over a balloon on a catheter, obviating the need to crimp the stent to the exterior of the balloon. At the target site, the balloon is inflated, causing the stent to partially unroll and expand to a cylindrical coil having a larger diameter with reduced overlap. In order to maintain the stent at the larger diameter, a locking or ratcheting mechanism is used. Some locking mechanisms comprise teeth on the edge of the sheet inside the coil that engage slots or holes in the adjacent wall of the stent. However, many locking mechanisms include an elongated tongue or belt that is attached to the inner edge of the coil and is drawn along the inner surface of the coil as it expands. In some configurations, the tongue or belt has a series of lateral ridges that engage with corresponding ridges on the inner wall of the stent and form a ratchet mechanism that maintains the stent at the enlarged diameter. Alternatively, the elongated tongue may have a series of holes that engage a corresponding series of projections on the interior wall of the coil and form a locking mechanism that keeps the stent at a fixed diameter. In either case, a portion of the tongue extends beyond the outer surface of the stent when the stent is tightly coiled.

Many cardiovascular delivery systems include a guide catheter in addition to the stent delivery catheter. In practice, the guide catheter is inserted into the patient's vascular system and advanced over a guide wire until the distal tip is adjacent to the target site. The stent delivery catheter is then passed through an interior lumen of the guide catheter. The guide catheter facilitates placement of the delivery catheter by providing a conduit having some longitudinal rigidity through the vascular system.

In order to deliver the stent, the distal portion of the delivery catheter bearing the stent is extended through distal tip of the guide catheter, and the stent is positioned at the target site. If it is necessary to reposition or replace the delivery catheter, the delivery catheter must be retracted into the guide catheter. However, in the tightly coiled configuration, the tongue portion of the ratchet mechanism extends beyond the inner diameter of the guide catheter, preventing its retraction into the guide catheter. A second problem encountered with guide catheters currently in use is that, due to their longitudinal rigidity, the catheters do not readily navigate through the vascular system and may cause an abrasion or dissection where the distal tip of the guide catheter contacts the vessel. It would be desirable, therefore, to provide a method and device for delivering a stent with a ratchet mechanism to a target site that would overcome these problems.

### SUMMARY OF THE INVENTION

One aspect of the invention provides a system for delivering a stent, comprising a delivery catheter having a movable elastic sleeve. A stent having a ratchet mechanism is positioned about the distal portion of the catheter and covered by the movable elastic sleeve. In a first position, the elastic sleeve covers the ratchet mechanism of the stent, and in the second position, the ratchet mechanism is uncovered.

Another aspect of the invention provides a method for treating a vascular condition and includes repositioning and deploying a stent having a ratchet mechanism at the treatment site. The distal tip of a guide catheter is advanced to an area adjacent to the treatment site. A delivery catheter carrying a stent having a ratchet mechanism, and covered by an elastic sleeve is advanced through the distal end of the guide catheter. During this procedure, the elastic sleeve prevents the stent from contacting the guide catheter or the vascular wall. Next, the delivery catheter is retracted back into the guide catheter. The elastic sleeve prevents the elongated tongue of the stent from protruding beyond the inner diameter of the guide catheter and preventing its retraction into the guide catheter. The guide catheter is then repositioned adjacent to a final target site. Next, the delivery catheter is advanced through the tip of the guide catheter, and positioned so that the stent is at the final target site. Finally, the elastic sleeve is retracted, and the stent is deployed precisely at the final target site.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE FIGURES

FIG. 1 is an illustration of a stent having a ratchet mechanism, as is known in the prior art;

FIG. 2 is an illustration of a guide catheter and delivery catheter bearing a stent having a ratchet mechanism, as is known in the prior art;

FIG. 3A is an illustration of a delivery system for a stent with a ratchet mechanism, in accordance with one aspect of the invention;

FIG. 3B shows the distal portion of the delivery system portrayed in FIG. 3A, in accordance with one aspect of the invention;

FIG. 4A is a side view of a guide catheter including a flexible tubular member adjacent to the distal tip of the guide catheter, in accordance with one aspect of the invention; and

FIG. 4B is an illustration of a guide catheter having a flexible tubular member, as the delivery catheter with a stent is advanced through the flexible tubular member, in accordance with one aspect of the invention; and

FIG. 5 is a flow diagram of a method of repositioning and deploying a stent having a ratchet mechanism within a vessel, in accordance with one aspect of the invention.

### DETAILED DESCRIPTION

Throughout this specification like numbers refer to like structures.

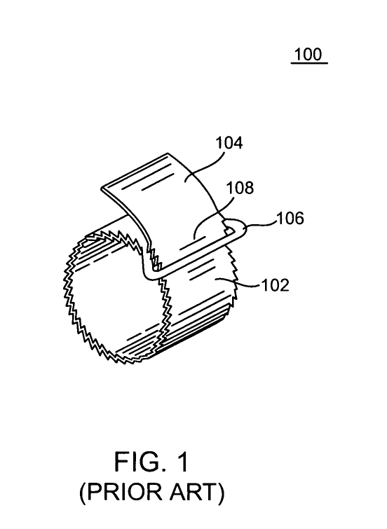

Referring to the drawings, FIG. 1 is an illustration of a stent 100 having a ratchet mechanism, as is known in the prior art. Such stents are formed by cutting a flat sheet of the stent material and rolling the sheet to form a spiral. The outer surface 102 of the spiral has a cylindrical shape. Stent 100 may be biodegradable or permanent (non-biodegradable), and is composed of a biocompatible material or a combination of biocompatible materials. Appropriate stent materials include metals, metal alloys such as stainless steel, shape memory materials such as nitinol, and biocompatible polymers such as polyetherketone, polymethylmethacrylate, polycarbonate, polyamide, polypropylene, polyethylene, polyethylene terephthalate, polyglycolide, polylactide, copolymers of lactide and glycolide, polyanhydrides, and other medically acceptable polymers, alone or in combination. The stent is deployed at the target site within the vascular system by inflating a balloon inside it, and causing the cylinder to expand and the walls to slide past each other, and form a cylinder with a larger diameter. Depending on the nature of the stent material, a ratcheting or locking mechanism is sometimes needed to prevent recoil and maintain the outer surface 102 of stent 100 in the cylindrical configuration having a larger diameter. Such locking mechanisms include at least one flexible, elongated tongue portion 104 that is attached to the spiral. Elongated tongue 104 passes through a slit or eye 106 on the exterior surface 102 of the stent 100, and when the stent 100 is tightly coiled, a portion of the tongue 104 extends beyond the outer surface 102 of the stent 100. As the stent 100 expands, the tongue portion 104 is drawn through the eye 106. In some configurations, the tongue portion 104 has a series of ridges 108 that engages with a portion of the eye 106 and forms a ratchet mechanism that maintains the stent 100 at the enlarged diameter.

The tongue portion 104 of the stent 100 comprises a biocompatible material that gives tongue portion 104 sufficient flexibility to enable it to slide through opening or eye portion 106 of the stent 100, but also sufficient rigidity to lock into place and support the stent 100 in the expanded configuration in the presence of the pressure exerted by the vessel wall. Consequently, when the stent 100 is in the tightly rolled configuration, the tongue portion 104 protrudes through the eye 106, and beyond the stent surface 102 and gives the stent 100 a larger effective diameter than it would otherwise have.

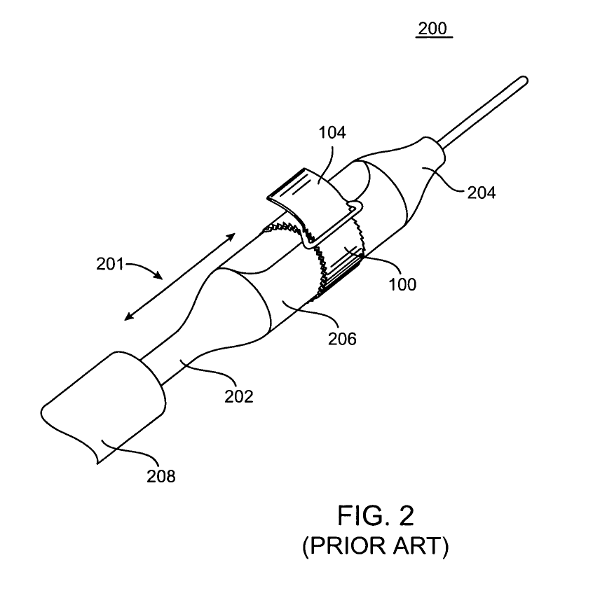

FIG. 2 is an illustration of delivery system 200, as is known in the art. The delivery catheter 201 includes a catheter shaft 202 having a tapered or rounded distal tip 204. Proximal to distal tip 204 is an inflatable balloon 206 shown in FIG. 2 in a collapsed configuration. A stent 100 is tightly rolled into a cylinder having a sufficiently small diameter so that when the stent 100 is placed over the balloon 206, it adheres firmly to the exterior surface of the collapsed balloon 206. As shown in FIG. 2, the distal portion of the delivery catheter 201 bearing the stent 100 is extended through distal tip of the guide catheter 208. Once the stent 100 is outside the guide catheter 208, the elongated tongue 104 of the stent ratchet mechanism protrudes beyond the inner diameter of the guide catheter 208, making it impossible to retract the delivery catheter into the guide catheter 208.

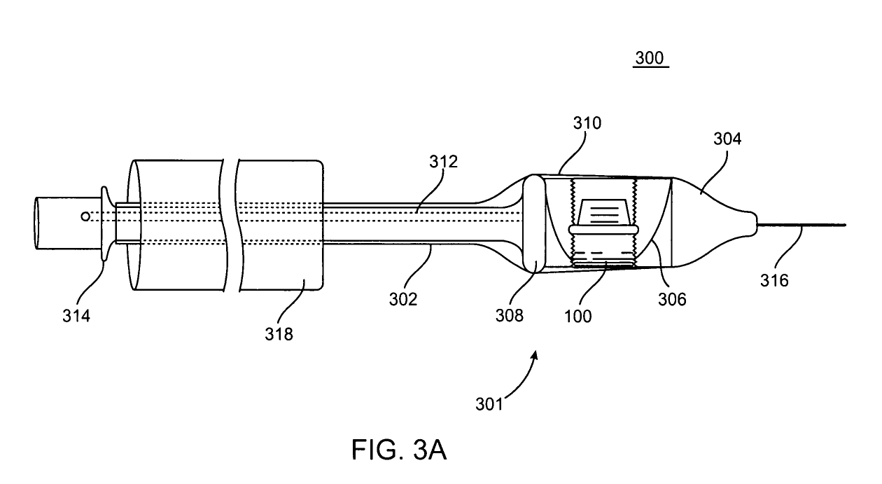

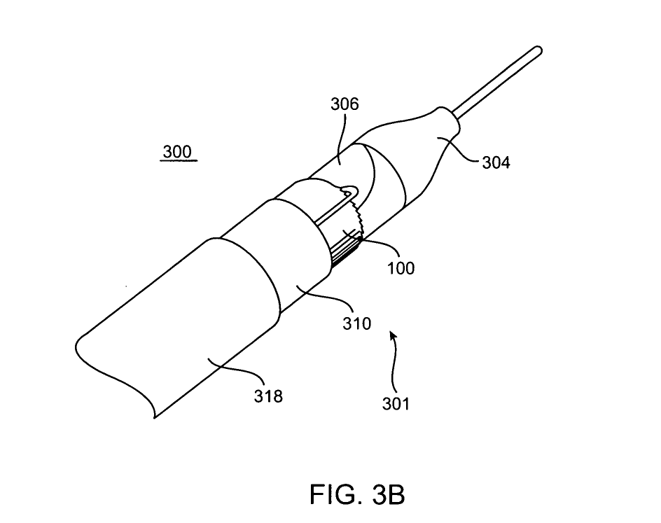

FIG. 3A shows a side view of delivery system 300 for stents having a ratchet mechanism, in accordance with one aspect of the invention. The delivery catheter 301 includes a catheter shaft 302 having a tapered distal tip 304. The catheter shaft 302 comprises a flexible, biocompatible polymeric material such as polyurethane, polyethylene, nylon, or polytetrafluroethylene (PTFE). In some embodiments, the delivery catheter 301 has a lumen that can accommodate a guide wire 316. The lumen runs longitudinally through the catheter 301, so that the delivery catheter 301 may be slipped over the guidewire, and, when no longer needed, the guide wire 316 may be withdrawn through the lumen of the catheter 301.

Proximal to distal tip 304 is an inflatable balloon 306 shown in FIG. 3 in a collapsed configuration. The balloon 306 comprises biocompatible, compliant, semi-compliant or non-compliant materials such as polyamides, polyurethanes, low density polyethylene, polyethylene terephthalate (PET), polyamide copolymers, polyurethane copolymers, and thermoplastic elastomers, as is presently known in the art. Balloon 306 is attached to the catheter body at the proximal and distal ends of the balloon by heat bonding, fusion bonding, adhesives or any other suitable means. The deflated balloon 306 is folded into longitudinal pleats, and wrapped around the catheter shaft. Balloon 306 is connected to a lumen 312 that extends through the delivery catheter 301 to the proximal end of delivery catheter 301. Balloon 306 is inflated by pumping a fluid through lumen 312 into the balloon, and thereby causing the longitudinal pleats to open, and the balloon 306 to expand. A stent 100 is tightly rolled into a cylinder having a sufficiently small diameter so that when the stent 100 is placed over the balloon 306, it adheres firmly to the exterior surface of the collapsed balloon 306.

In one embodiment of the invention, balloon 306 includes a proximal end portion 308 that has a diameter that is larger than the inner diameter of the stent 100 in the tightly rolled configuration. The enlarged proximal end portion 308 of the balloon 306 may be a ring around the catheter body or a pillow of a flexible polymeric material. The enlarged portion 308 maintains the stent 100 in position over the wrapped balloon 306 and prevents the stent 100 from sliding in a proximal direction along the catheter shaft 302.

In one embodiment of the invention, a cylindrical elastic sleeve 310 extends from the proximal end of the tapered distal portion 304 of the delivery catheter 301 and surrounds the stent mounting portion of delivery catheter 301 including at least a portion of the stent 100 and wrapped balloon 306. The elastic sleeve 310 comprises thermoplastic elastomers having an optimal elongation index and flexibility, latex, and natural or synthetic rubber, or any other suitable material. Elastic sleeve 310 is sized and positioned so that it is slightly stretched over the exterior of stent 100 including the tongue portion, and holds the tongue portion against the exterior surface of the stent 100. Consequently, the elastic sleeve 310 prevents the tongue portion from extending away from the stent 100, and preventing retraction of the delivery catheter 301 into the guide catheter 318. However, catheter 301 may also be used to deliver stents that do not have a ratchet mechanism. For example, in one embodiment of the invention, the external surface of elastic sleeve 310 is coated with a lubricious substance such as silicone, polytetrafluroethylene (PTFE), or a hydrophilic coating. In this embodiment, elastic sleeve 310 provides the delivery catheter 301 with a low profile and a uniform, smooth lubricious exterior surface, providing an advantageous delivery system for stents of various designs.

In one embodiment of the invention, tubular sleeve 310 extends to the proximal end of delivery catheter 301. In this embodiment, the sleeve is retracted by pulling the proximal end 314 of the sleeve 310 so that the stent 100 is exposed to the interior of the blood vessel. As the elastic sleeve 310 is withdrawn, enlarged portion 308 prevents the stent 100 from being drawn by the elastic sleeve 310 in a proximal direction along the catheter shaft 302. In one embodiment of the invention, the interior surface of the elastic sleeve 310 is coated with a lubricious material such as silicone, polytetrafluroethylene (PTFE), or a hydrophilic coating. The lubricious interior surface of the elastic sleeve facilitates retraction of the sleeve 310 so that it readily slides over the stent 100 without dislodging it from the catheter shaft 302.

FIG. 3B is an external side view of the distal portion of delivery system 300. The distal portion of delivery catheter 301 including the tip 304 and the adjacent stent mounting area have been advanced through the distal end of the guide catheter 318. Portions of the wrapped balloon 306 and of the stent 100, including the ratchet mechanism, are covered by elastic sleeve 310. The elongated tongue portion 104 of the stent 100 is retained within the elastic sleeve 310, and does not extend beyond the inner diameter of the guide catheter 318. In this embodiment of the invention, the delivery catheter 301 can easily be retracted into the guide catheter 318.

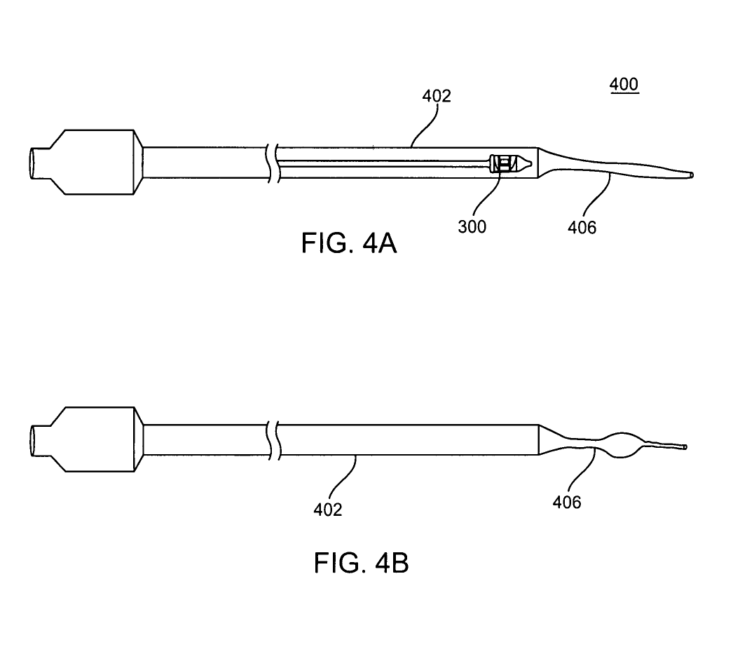

FIG. 4A is a side view of a guide catheter 400 that, in one embodiment of the invention, is used in conjunction with delivery catheter 300 illustrated in FIG. 3. The body 402 of catheter 400 is a hollow tubular structure comprising a flexible, biocompatible polymeric material such as polyurethane, polyethylene, nylon, or polytetrafluroethylene (PTFE), or any other suitable material. In one embodiment, guide catheter 400 has a lumen that runs longitudinally through catheter body 402 and can accommodate a guide wire. The guide catheter 400 is slipped over the guide wire, and guided along the vascular route, until the distal portion of the guide wire and guide catheter 400 are at their desired target locations. The stent delivery catheter 300 is then advanced through the interior lumen of the guide catheter 400 to the treatment site. The guide catheter body 402 has sufficient flexibility to accommodate sharp bends in the vascular system, but also has sufficient longitudinal rigidity to enable it to pass through narrow stenotic lesions.

In one embodiment of the invention, a flexible tubular member 406 is attached to the distal end of catheter body 402. Tubular member 406 comprises a flexible, pliable material such as a deformable elastomer, silicone rubber, polyester fabric, or other suitable materials. In one embodiment of the invention, the external surface of tubular member 406 is coated with a lubricious material such as silicone, polytetrafluroethylene (PTFE), or a hydrophilic coating. Tubular member 406 facilitates passage of guide catheter 400 through sharp bends, branch points, and ostia of the vascular system. As guide catheter 400 is pushed forward through the vascular system, if the curved tubular member 406 engages an impediment within the vessel, it will slide over the impediment, or bend and allow the guide catheter to by-pass the impediment. In one embodiment of the invention, the interior surface of the tubular member 406 is coated with a lubricious material such as silicone, polytetrafluroethylene (PTFE), or a hydrophilic coating. In combination with the lubricious exterior surface of the elastic sleeve 310 on the delivery catheter, the lubricious surface of tubular member 406 facilitates retraction of the distal portion of the delivery catheter 300 through tubular member 406. FIG. 4A shows the delivery catheter 300 placed in the distal portion of the guide catheter 400 prior to deployment.

FIG. 4B presents an external view of guide catheter 400 as the delivery catheter is advanced through tubular member 406. Tubular member 406 has sufficient elasticity to expand over the balloon portion of the delivery catheter during its passage through tubular member 406, and allow deployment of the distal portion of the delivery catheter to the delivery site in the blood vessel.

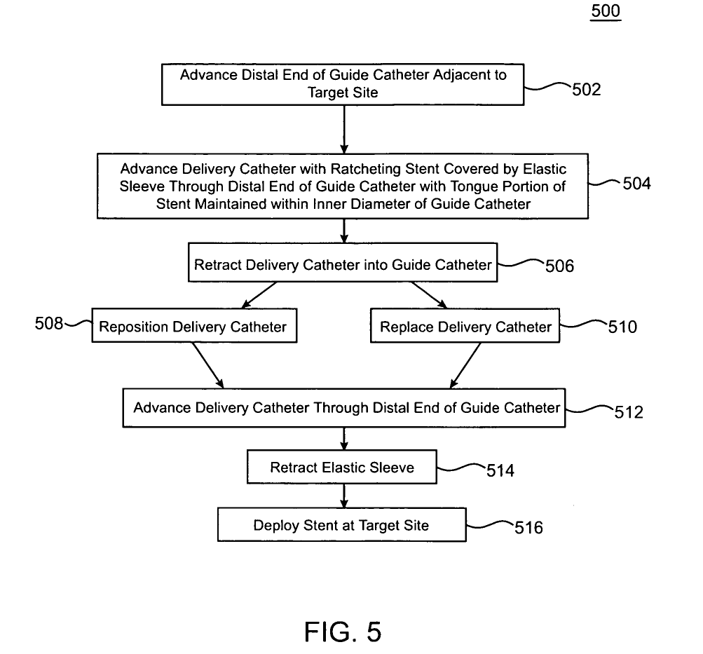

FIG. 5 is a flow diagram illustrating a method 500 for delivering a stent having a ratchet mechanism to a target site in the vascular system. The method begins wherein the distal end of the guide catheter is advanced through the vascular system and placed adjacent to the target site (Block 502). A guide wire may be used to guide the catheter through the vascular system. The guide wire is inserted into the femoral vein, the jugular vein, subclavian vein, or other point of access, depending upon the location of the lesion to be treated. Guide catheter 400, shown in FIG. 4, is then slipped over the guide wire and guided through the vascular system until the distal tip of the catheter arrives at the target site. The flexible tubular member 406 at the distal tip of guide catheter 400 facilitates its passage through the vascular system. The procedure may be visualized using fluoroscopy, echocardiography, intravascular ultrasound, angioscopy, or other means of visualization.

Next, the delivery catheter 301, shown in FIG. 3, is passed through the guide catheter 400 until the distal tip of the delivery catheter is adjacent to the distal end of the guide catheter. Near the distal tip of the delivery catheter, a tightly rolled stent of the type shown in FIG. 1 is mounted on the exterior surface of a balloon. The stent is covered by an elastic sleeve that is stretched over the exterior surface of the stent, including the tongue of the ratchet mechanism. The distal portion of the delivery catheter is advanced through the distal end of the guide catheter (Block 504). Because the stent is covered by the elastic sleeve, the tongue of the ratchet mechanism is held close to the stent, and the tongue cannot contact the vessel wall.

It is sometimes impossible to place the stent precisely at the target site, making it necessary to retract the delivery catheter into the guide catheter (Block 506) and then reposition the delivery catheter (Block 508). The elongated tongue portion of the stent is retained within the elastic sleeve so that the tongue portion of the ratchet mechanism does not extend beyond the inner diameter of the guide catheter, preventing its retraction into the guide catheter. Additionally, the retraction process is facilitated by the smooth lubricious exterior surface of the elastic sleeve covering the stent, and the lubricious interior surface of the guide catheter.

It is sometimes discovered during the course of the procedure that the stent is not the correct size, or of optimal design to treat the lesion. In such circumstance, the delivery catheter may be withdrawn and replaced with a delivery catheter (Block 510) and, if appropriate, a stent more suitable for the patient.

Next, the delivery catheter is once again advanced through the distal end of the guide catheter (Block 512). The delivery catheter is manipulated so that the stent is placed precisely at the target site. The manipulation of the delivery catheter is facilitated by the low profile and smooth lubricious exterior surface of the distal portion of the delivery catheter provided by the elastic sleeve that fits tightly over the balloon and stent, and covers the ratchet mechanism of the stent.

With the stent placed precisely at the target site, the elastic sleeve is retracted by pulling the proximal end of the elastic sleeve (Block 514). The retraction process is facilitated by the lubricious interior surface to the elastic sleeve. The stent is held in place by the enlarged pillow at the proximal end of the balloon as the elastic sleeve slides over the stent. Next, the stent is deployed at the final target site by expanding the balloon on the delivery catheter (Block 516). The stent is expanded by the balloon, and is held in the expanded configuration by the ratchet mechanism. Finally, both the delivery catheter and the guide catheter are withdrawn from the body.

While the invention has been described with reference to particular embodiments, it will be understood by one skilled in the art that variations and modifications may be made in form and detail without departing from the spirit and scope of the invention.

What is claimed is:

1. A system for delivering a stent to a target site in a vessel, comprising:
a delivery catheter;
a stent disposed on the distal portion of the delivery catheter, the stent having a ratchet mechanism; and
an elastic sleeve positioned over the stent, the elastic sleeve moveable from a first position to a second position, wherein in the first position the ratchet mechanism is covered by the elastic sleeve and in the second position the ratchet mechanism is uncovered.

2. The system of claim 1 wherein the stent comprises one or more polymeric materials.

3. The system of claim 1 wherein the ratchet mechanism comprises an elongated tongue portion of the stent disposed through an eye portion of the stent.

4. The system of claim 3 wherein the elongated tongue portion of the stent is retained within the elastic sleeve prior to deployment of the stent at a target site.

5. The system of claim 1 wherein the elastic sleeve fits tightly over at least a portion of the stent.

6. The system of claim 1 wherein the elastic sleeve may be retracted prior to deployment of the stent at a target site.

7. The system of claim 1 wherein the elastic sleeve provides a smooth exterior surface to the delivery catheter prior to deployment of the stent.

8. The system of claim 7 wherein the smooth exterior surface of the delivery catheter facilitates longitudinal movement of the delivery catheter in relation to a guide catheter.

9. The system of claim 7 wherein the exterior surface of the elastic sleeve is coated with a lubricious material selected from the group consisting of silicone, polytetrafluroethylene (PTFE), a hydrophilic coating, and any combination thereof.

10. The system of claim 1 wherein the elastic sleeve comprises one or more materials selected from the group consisting of thermoplastic elastomers, rubber, and latex.

11. The system of claim 1 wherein the interior surface of the elastic sleeve is coated with a lubricious material selected from the group consisting of silicone, polytetrafluroethylene (PTFE), a hydrophilic coating, and any combination thereof.

12. The system of claim 1 further comprising an expandable balloon attached to the distal portion of the delivery catheter wherein the balloon is expanded to deploy the stent at a target site.

13. The system of claim 12 wherein a proximal portion of the balloon has a diameter that is larger than the inner diameter of the stent, to thereby retain the stent on the distal portion of the delivery catheter prior to deployment of the stent.

14. The system of claim 1 wherein the target site is an ostium of a cardiac artery.

15. The system of claim 1 further comprising a guide catheter having a flexible tubular member positioned adjacent to the distal tip thereof wherein the flexible tubular member facilitates passage of the delivery system through the vessel.

16. The system of claim 15 wherein the interior surface of the flexible tubular member is coated with a lubricious material selected from the group consisting of silicone, polytetrafluroethylene (PTFE), a hydrophilic coating, and any combination thereof.

17. A method of repositioning and deploying a ratcheting stent within a vessel, the method comprising:
advancing a distal end of a guide catheter adjacent to a target site within the vessel;
advancing a delivery catheter having a ratcheting stent disposed on the distal portion thereof, and having an elastic sleeve positioned thereover, through the distal end of the guide catheter, wherein the elastic sleeve maintains a tongue portion of the stent within an inner diameter of the guide catheter;
retracting the delivery catheter into the guide catheter;
repositioning the delivery catheter adjacent to a final target site;
re-advancing the delivery catheter through the distal end of the guide catheter;
retracting the elastic sleeve; and
deploying the stent at the final target site.

18. The method of claim 17 wherein advancing the distal end of the guide catheter to an area within the vascular system adjacent the target site is facilitated by a flexible tubular member adjacent the distal end of the guide catheter.

19. The method of claim 17 further comprising advancing the delivery catheter through a flexible tubular member adjacent to the distal end of the guide catheter.

20. The method of claim 17 wherein deploying a stent further comprises inflating an expandable balloon attached to a distal portion of the delivery catheter.

21. The method of claim 17 further comprising retracting the delivery catheter through a flexible tubular member adjacent the distal end of the guide catheter.

22. The method of claim 17 wherein the target site is an ostium of a cardiac artery.

23. The method of claim 17 wherein an elongated tongue portion of the stent is retained within the elastic sleeve prior to deployment of the stent at the final target site.

24. The method of claim 17 wherein advancing the delivery catheter through the distal end of the guide catheter is facilitated by a smooth lubricious exterior surface of the elastic sleeve.
